(19) United States
(12) Patent Application Publication
(10) Pub. No.: US 2012/0059483 A1
(43) Pub. Date: Mar. 8, 2012

(54) ORTHOPEDIC INTERFACE DEVICE AND METHOD

(75) Inventors: E. Skott GREENHALGH, Lower Gwynedd, PA (US); John-Paul ROMANO, Chalfont, PA (US)

(73) Assignee: STOUT MEDICAL GROUP, L.P., Perkasie, PA (US)

(21) Appl. No.: 13/293,921

(22) Filed: Nov. 10, 2011

Related U.S. Application Data
(63) Continuation of application No. PCT/US2010/038554, filed on Jun. 14, 2010.
(60) Provisional application No. 61/186,695, filed on Jun. 12, 2009.

(51) Int. Cl. A61F 2/38; A61F 2/30

(52) U.S. Cl. 623/20.11; 623/18.11; 623/20.14

(57) ABSTRACT

An orthopedic joint implant component and implant component surface are disclosed. The implant component can have a resilient or compliant structure to distribute force loads. The implant component surface can be attached by one or more springs or other resilient or compliant elements to the remainder of the implant. Methods of using the component and component surface are also disclosed.

### CROSS-REFERENCE TO RELATED APPLICATIONS

This application is a continuation of PCT Application No. PCT/US2010/038554, filed 14 Jun. 2010, which claims priority to U.S. Provisional Application No. 61/186,695, tiled 12 Jun. 2009, which are both incorporated by reference herein in their entireties.

### BACKGROUND OF THE INVENTION

1. Field of the Invention

Orthopedic interface devices and methods for use are disclosed. More specifically, devices for use in joints to more closely mechanically simulate natural joint mechanics are disclosed.

2. Description of Related Art

FIG. 1 illustrates a knee 1 with a typical bilateral knee implant 2. The bilateral knee implant 2 is also known as a total knee implant or prosthesis. The bilateral or total knee implant 2 can replace the surface of both the medial and lateral condyles of the femur 4, the entire corresponding surface at the proximal end of the tibia 6 and the entire meniscus.

The knee 1 implant includes a solid knee implant femoral component 8 at the distal end of the femur 4 and a tibial component 10 at the proximal end of the tibia 6. The knee implant femoral component 8 and tibial component 10 are often a hard metal, such as steel, or a cobalt chrome alloy. The knee implant femoral and tibial components 8 and 10 are intended to simulate the respective ends of the bones. However, the knee implant femoral and tibial components 8 and 10 are made of rigid materials used for toughness and durability, but do not cushion the absorption of an impact force similar to a natural knee or provide an ideal rotational surface.

A meniscus component 12, bearing surface or bearing component, is often attached to the proximal side of the tibial component 10. The meniscus component 12 is intended to simulate the cartilage and is often made of a softer material than the knee implant femoral component 8 and tibial component 10. The meniscus component 12 can be made from a polymer, such as ultra high molecular weight polyethylene, PTFE or PET.

During use of the knee 1 and the knee implant 2, the tibial actual plane 14 at the top surface of the tibia 6 can be rotated from the tibial natural plane 16 at the top surface of the tibia 6. This rotation can occur when the patient is at rest or during activity. The rotation can result in the load of the knee 1 shifted to one side of the knee 1, shown as the medial side in FIG. 1. Because the components of the knee implant 2 do not closely enough mimic the natural tissue, a stress riser 18 or area of higher mechanical stress concentration can occur on the medial side of the knee 1. The stress riser 18 can result in accelerated wear of the implant components, most notably the bearing component (i.e., meniscus component 12), but also the knee implant femoral component 8 and the tibial component 10. The stress riser 18 can result in bone loss due to high loads and implant breakage. Furthermore, implants 2 can be cemented in place, for example with bone cement, such as PMMA. High stresses can break or chip PMMA cement resulting in partial or complete failure of the components and/or surrounding tissue (e.g., pain and broken bones).

The stress riser 18 and/or the mismatch of the mechanical characteristics of the implants to the natural tissue can also result in stress risers 18 between the components and the surrounding tissue. For example, stress risers 18 around the tibial stem 20, which can anchor the tibial component 10 within the tibia 6, can separate from tibia 6, and/or break or otherwise damage the tibia 6.

FIG. 3 illustrates a knee 1 with unilateral damage to the cartilage of the knee 1. As shown, the knee 1 can have lateral condyle femoral cartilage 22a and lateral meniscus cartilage 24a that can be thicker and in better condition than the medial meniscus cartilage 24b and possibly the medial condyle femoral cartilage 22b, which can be worn down resulting in unilateral osteoarthritis.

FIG. 4 illustrates a knee 1 with a unilateral knee prosthesis or implant 2. The unilateral prosthesis or implant 2 can replace the surface of a single condyle, such as the medial condyle as shown, of the femur 4 and the corresponding side of the tibia 6, and meniscus 24. Similar to the total (i.e., bilateral) knee implant, the medial condyle and medal tibial components 8b and 10b are single pieces of rigid, substantially inflexible, hard material.

When the knee 1 is in a natural, healthy condition, the femur 6 has a femoral natural longitudinal axis 26b aligned at a natural angle with respect to the longitudinal axis of the tibia. After an implant 2 is inserted into the knee 1, especially a unilateral implant but also with a total knee implant, the femur actual longitudinal axis 26a can be offset from the femoral natural longitudinal axis 26b, as shown by arrow 28. This rotation of the femur 4 relative to the tibia 6 (or tibia 6 relative to the femur 4 depending on the reference location) can be a result of an inappropriately sized or positioned implant 2. For example, the doctor can remove the incorrect amount of bone for the location in which the implant 2 is to be deployed. As shown in FIG. 4, the medial meniscus component 12b can be too large, resulting in lateral rotation of the proximal end of the femur 4. This resulting unnatural position can alter the patient's gait, produce damage around the knee implant 2 (such as stress risers 18, as explained above), and also in other

FIG. 5 illustrates a knee implant 2 with a medial meniscus component 12h that is too small, resulting in the proximal end of the femur 4 (or tibia 6 depending on the reference location) rotating, as shown by arrow 28, unnaturally in the medial direction. This unnatural rotation can result in the same biomechanical problems as described above. The surrounding femoral condyle component 8b and tibial component 10b are rigid and inflexible and the mechanics of the components are not adjustable to mitigate damage caused by components that are not properly sized.

FIG. 7 illustrates a typical tibia component 10 that has a tibial plate 30 and a tibial stem 20. The tibial plate 30 is shown with distributed load forces 32 applied. As shown, the tibial plate 30 is not deforming to accommodate the distribution of the plate load forces 32. If the load forces 32 spike at a load riser or stress riser 18 at any location on the tibial plate 30, the tibial component 10 can not deform to distribute the pressure.

FIG. 10 illustrates a hip implant femoral component 34. The hip implant femoral component 34 can have a femoral stem 36 and a neck 38. The hip implant femoral component 34 is typically made of a rigid, inflexible structure. The distributed load forces 32 around the hip implant femoral component 34 can result in stress risers 18 and the associated problems with stress risers 18, as described above. The hip implant femoral component 34 can break from the femur 4 and/or break the femur 4 itself after implantation during use.

Therefore, an orthopedic implant is desired that can adjust to distribute forces to minimize stress risers around the implant.

### SUMMARY OF THE INVENTION

A joint component surface of an orthopedic implant is disclosed. The component surface can distribute stress risers. The joint surface can be resiliently attached to or integral with the remainder of the component. For example, the joint surface can have a coil and/or leaf spring between the component surface and the stem anchoring the implant in the bone.

An implantable artificial joint component is disclosed that can resiliently deform. The joint component can have a spring within the body of the joint. For example, the joint component can be a tibial component for a knee implant. The tibial component can have a base plate attached to a top plate by one or more plate springs or struts.

The component or component surface can be implanted in joints, such as in the hip, knee, elbow, liners, toes, spine (e.g., between vertebrae to aid in fusing adjacent vertebral bodies), or for non-joint applications, such as to fix a long bone break, or the repair of a surgical opening such as a broken sternum.

### BRIEF DESCRIPTION OF THE DRAWINGS

FIG. 1, not the invention, is an anterior view of a knee having a typical knee implant.

FIG. 2 is an anterior view of the knee with a deployed variation of the disclosed device.

FIG. 3, not the invention, is an anterior view of a knee and associated cartilage.

FIGS. 4 and 5, not the invention, are anterior views of the knee of FIG. 3 with various typical knee implants.

FIG. 6 is an anterior view of the knee of FIG. 3 with a deployed variation of the disclosed device.

FIG. 7, not the invention, illustrates a stress loaded typical tibial component.

FIG. 8a illustrates a stress loaded disclosed tibial component.

FIGS. 8b and Sc are variations of close up A-A of FIG. 8a at first and second time points, respectively, during a stress load.

FIG. 9 illustrates a variation of the femoral component of a bilateral knee implant.

FIG. 10, not the invention, illustrates a typical hip implant femoral component.

FIGS. 11 through 14 are variations of the disclosed hip implant femoral component.

FIGS. 15a through 15d are perspective, top, bottom, and side views, respectively, of a variation of the acetabulum component.

FIGS. 15e is a variation of a side view of cross-section B-B.

FIG. 15f is a perspective view of a variation of cross-section B-B.

FIG. 15g is a perspective view of a variation of cross-section B-B at a different angle than the view of FIG. 15f.

FIG. 16 is an anterior x-ray visualization of the deployed unilateral knee implant.

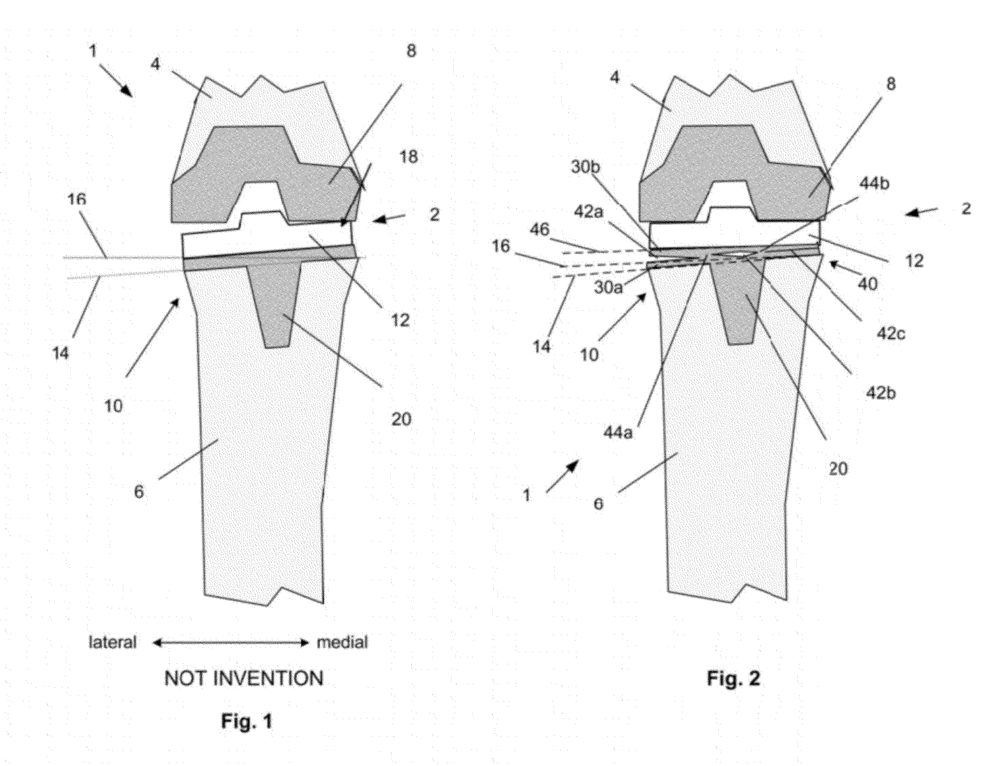

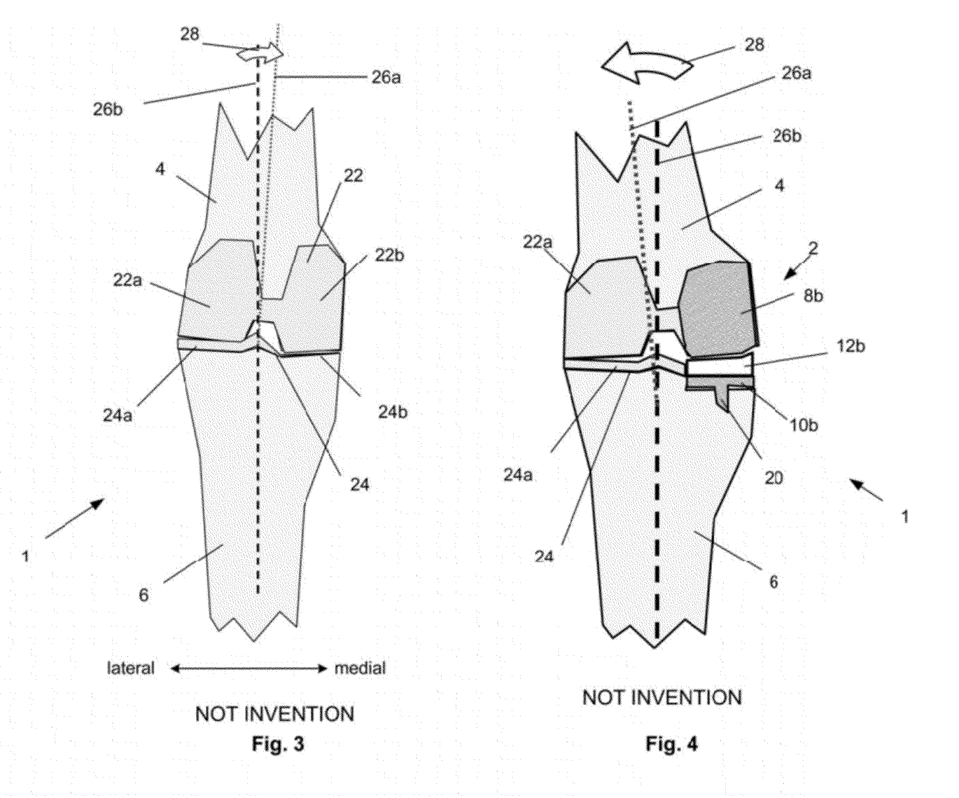

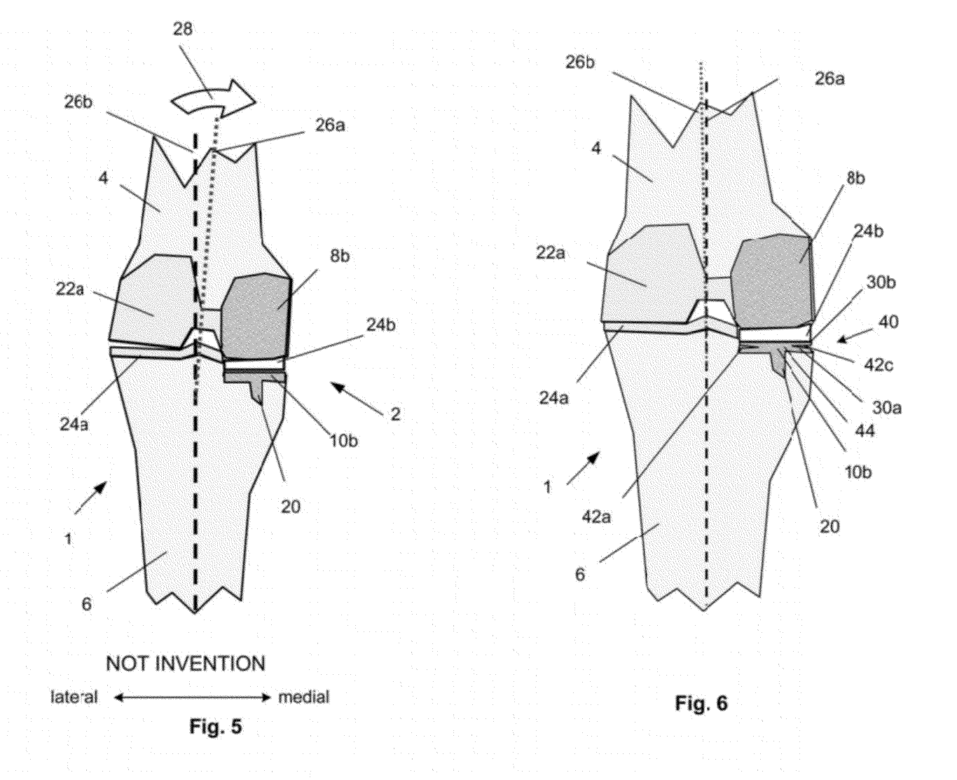

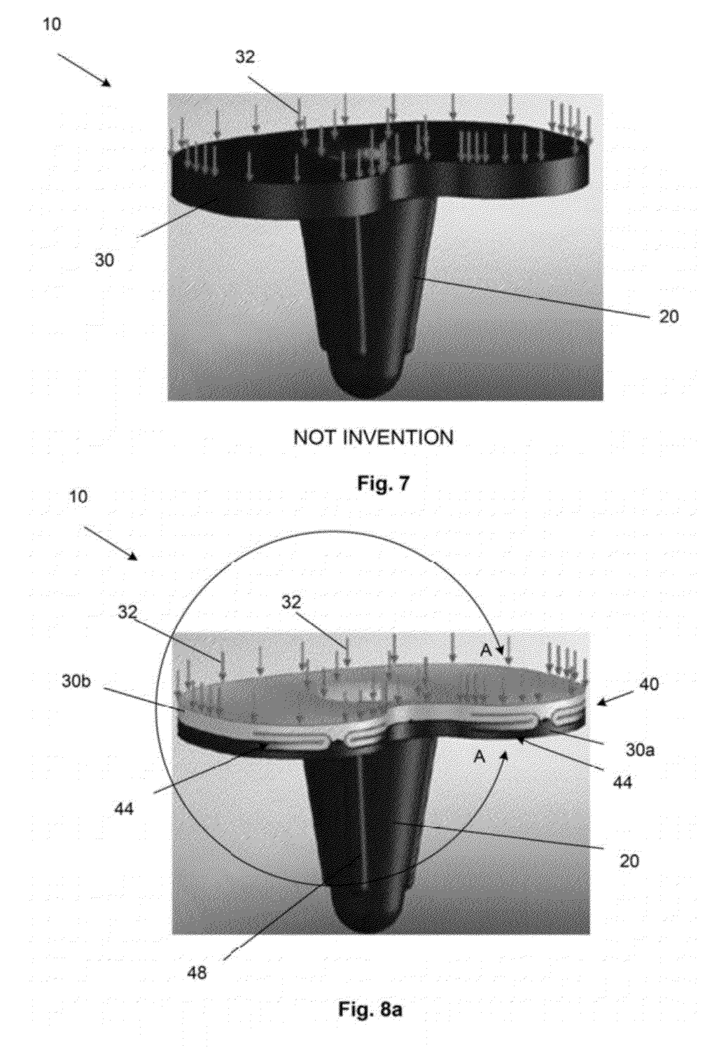

### DETAILED .DESCRIPTION

FIG. 2 illustrates that a device, such as a joint or knee implant 2 can have a tibial component 10 that can have a tibial base plate 30a resiliently attached to a tibial top plate 30b. The tibial top plate 30a can be attached to the meniscus or hearing component 12. The tibial top plate 30b can form the outer surface of the tibial component 10. The tibial base plate 30a can be fixedly or resiliently attached to or integral with the tibial stem by one or more plate springs 40.

The springs 40 can have a damping or dampening coefficient. The damping coefficient can be related to the spring coefficient. The damping coefficient can be a well damped to over damped ratio to the spring coefficient, for example resulting a few oscillations (e.g., less than about 10, or more narrowly less than about four) to return to equilibirum. For example, the spring can reset to the original position before the next heel-strike or foot-strike during walking (about 1 sec to about 2 sec) or running (about 0.3 sec to about 0.8 sec).

The springs 40 can have a relaxed length and a minimum length of travel. At the minimum length of travel, the spring 40 can be completely compressed between the tibial top plate 30a and the tibial bottom plate 30h.

The springs 40 can be submerged in a biological fluid (e.g., synovial fluid, blood) or non-biological fluid (e.g., saline solution) after delivery to a target site. The springs 40 can have enclosed volumes, such as with bellows, or quasi-contained volumes, for example hounded by the tibial base plate 30a and tibial top plate 30b. The springs 40 can act as visco-elastic dampers (or dampeners). The fluid can be compressed and/or drawn in (e.g., refilled) and expunging out of the spring 40 during expansion and contraction of the spring 40. The compression or drawing and expunging of fluid in the spring 40 can create a mechanical damping (or dampening) effect of the spring 40. The fluid dampening effect can occur in any of the variations (e.g., unilateral knee, bilateral knee, hip stem, acetabular cup, vertebra). The fluid dampening effect can be changed by changing the fluid viscosity (e.g., by injecting saline into the joint capsule) and rate of expansion and contraction of the spring 40 (e.g., heavier dampening will occur with the knee implant 2 during running than walking).

The tibial stem 20 and/or tibial base plate 30a can have one or more ingrowth matrices configured to induce bone growth into the component to anchor the component to the surrounding bone after delivery to the target site. The tibial stem 20 and/or tibial base plate 30a can be cemented in place with PMMA.

The plate springs 40 can be flat springs, such as leaf springs such as full elliptical. semi elliptical or quarter-elliptical springs, non-elliptical, parabolic leaf springs, or combinations thereof The plate springs 40 can be torsion springs, such as a spiral mainspring. The plate springs 40 can be a compression spring, such as coil or helical springs, belleville springs or washers, volute springs, spring washers such as curved or wave washers or slotted or finger washers, gas springs, or combinations thereof. The plate springs 40 can be cross-struts or cantilever or beam springs. The plate spring force can be a result of the tibial top plate 30b or tibial base plate 30a being magnetized or electro-magnetically charged and the opposite plate (i.e., the tibial top 30b or base 30a) being similarly magnetized or electro-magnetically charged. The plate springs 40 can be a combination of one or more of the springs described above.

The tibial component 10 can have a cell and strut configuration integral with the tibial top plate 30b and the tibial base plate 30a. The cell and strut configuration can have a lateral cell 42a, a central cell 42b and a medial cell 42c. The tibial component 10 can have a lateral strut 44a between the lateral cell 44a and the central cell 44b. The tibial component 10 can have a medial strut 44b between the medial cell 44c and the central cell 44b. The struts 44 can be the springs 40.

The tibial top plate 30b can be rotated and translated with respect to the tibial base plate 30a. When unevenly distributed load forces 32 are applied to the tibial top plate 30b, the tibial top plate 30h can rotate to more evenly distribute the load force 32 on the top plate 30b, for example reducing the maximum stress to the tibial top plate 30b, the meniscus component 12, the knee implant femoral component 8, and the surrounding tissue in the femur 4, tibia 6 and elsewhere in the body. The rotation of the tibial top plate 30b can reduce stress risers 18. The tibial top plate plane 46 can be substantially parallel and/or equal to the tibial natural plane 16 during use, for example during uneven lateral force loading of the knee 1 as shown in FIG. 2. The tibial top plate plane 46 can be non-parallel or parallel with the tibial actual plane 14.

Under typical dynamic loads (i.e., activity) the implant spring 40 can absorb energy and reduce impact type loads. The implant spring 40 can act as a cushion (e.g., a damper and/or a spring). The implant spring 40 can reduce the bearing surface impingement failure. Bearing surface impingement failure can occur when the bearing surface (e.g., UHMWPE) is pinched between the stronger, stiffer tibial and femoral components 10 and 8. The pinching can cause high subsurface stresses on the bearing component and UHMWPE internal damage.

FIG. 6 illustrates that the medial tibial component 10b can cover about half or less than half of the tibial proximal surface. The medial tibial component 10b can have the tibial top plate 30b connected, integral with or attached to the tibial base plate 30a by one or more struts 44 and/or other configurations of plate springs 40. The tibial top plate 30b can rotate and/or translate with respect to the tibial base plate 30a as described supra.

The femoral natural longitudinal axis 26b can be substantially equal to the actual femoral longitudinal axis 26a. The strut 44 or base spring 40 can resiliently deform to accommodate translation and rotation of the components of the implant 2, such as the tibial component 10, the femoral component 8 (e.g., medial condyle component 8b), the meniscus component 12 (e.g., the medial meniscus component 12b), or combinations thereof. The surface of the tibial top plate 30a can remain in substantially constant and even contact with the surface of the meniscus component 12.

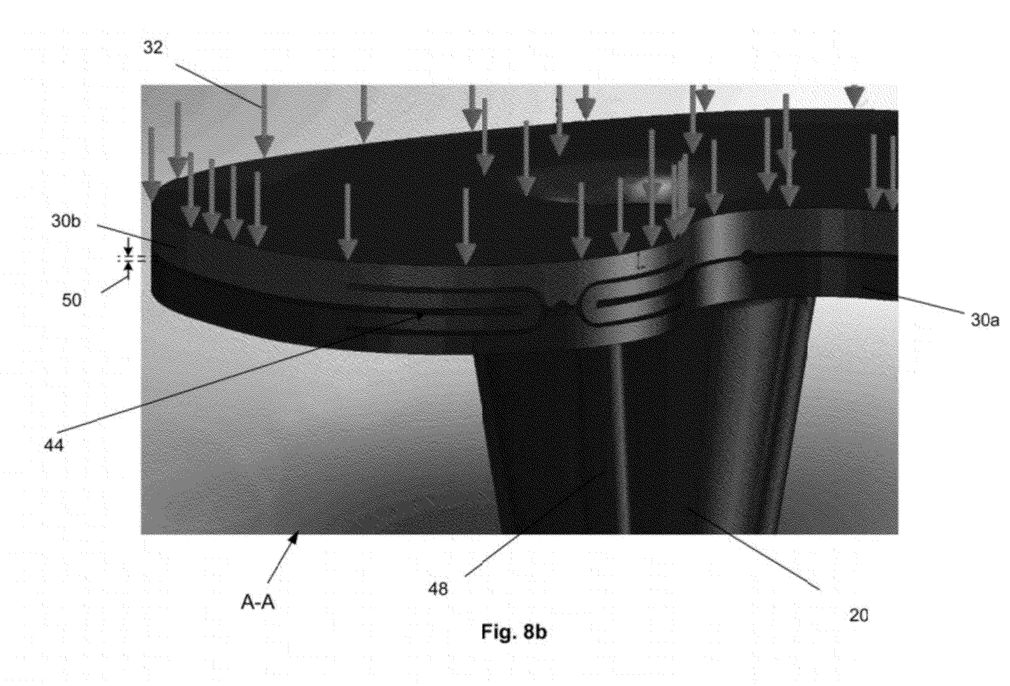

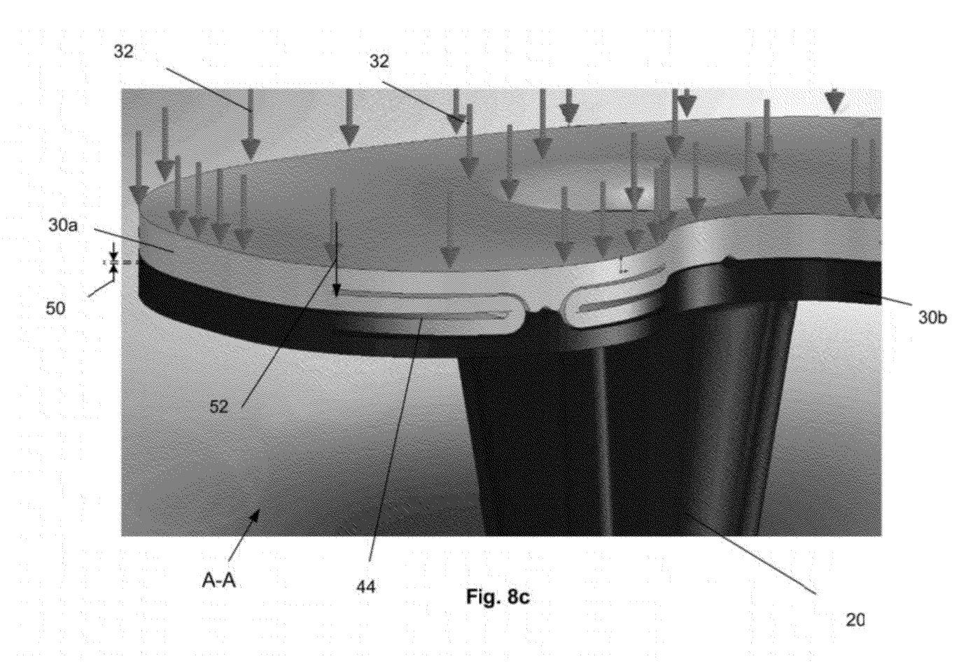

FIG. 8a illustrates that the struts 44 can resiliently deform under load forces 32. The tibial top plate 30b can translate and rotate when the struts 44 deform.

The deformation of the struts 44 and the axial translation and/or rotation of the top plate 30b with respect to the base plate 30a can reduce the maximum pressures or stresses from the impact load forces 32 applied on the tissue and other implant components surrounding the tibial component 10 and the proximal shelf of the tibia bone 6. The reduction of stresses caused by impact forces can reduce implant loosening, migration, and bone loss.

The struts 44 can be symmetrically located about a longitudinal axis through the tibial stem 20. The implant 2 can have four medial struts 44b and four lateral struts 44a.

The tibial stem 20 can have one or more (e.g., four) tibial stem ribs 48 extending radially from the tibial stem 20. The tibial stem rib 48 can rotationally and/or axially anchor or fix the tibial component 10 in the tibia 6. The tibial stem ribs 48 can have unidirectional teeth or barbs.

FIGS. 8b illustrates the tibial component 10 during initial loading, for example when the leg is not bearing a significant force. The tibial top plate 30h can be spaced from the tibial base plate 30a by a plate gap 50. The plate gap 50 can be from about 0.05 mm (0.002 in.) to about 0.381 mm (0.015 in.), for example about 0.05 mm (0.002 in.).

The configuration of the plate gap 50 can effect the fluid dampening characteristics of the implant 2. For example, if the cells 42 have a more closed configuration, the fluid entering and exiting the cells will experience higher flow resistance, resulting in a higher dampening effect and vice versa. If the cells 42 or slots have more turns or are more tortuous, or have additional obstacles to the flow (e.g., baffles, leaflets, shrouds, valves, of combinations thereof) the fluid dampening effect can be increased.

FIG. 8c illustrates that the tibial top plate 30b can translate, as shown by top plate translation arrow 52, toward the tibial base plate 30a during loading. The plate gap 50 can reduce to about 0. The force load 32 delivered to the tibial top plate 30h can be from about 2 to about 5 times the body weight of the patient, for example from about 800 N (180 lbs.) to about 6.7 kN (1,500 lbs.). When subjected to about 5 times the expected body weight of the patent, the tibial component 10 can have a deformation such that the plate gap 50 can be reduced, but greater than about 0. The struts 44 or plate springs 40 can deform so the struts 44 concurrently abut or are contact with the adjacent plates 30a and 30b. As the load force 32 is reduced, the struts 44 or plate springs 40 can deform away from where the struts 44 concurrently abut or contact with the adjacent plates 30a and 30b.

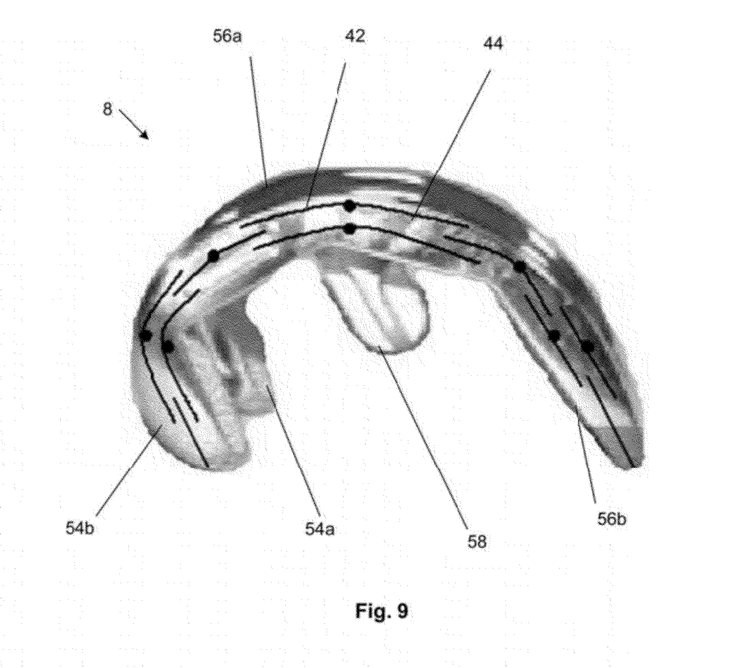

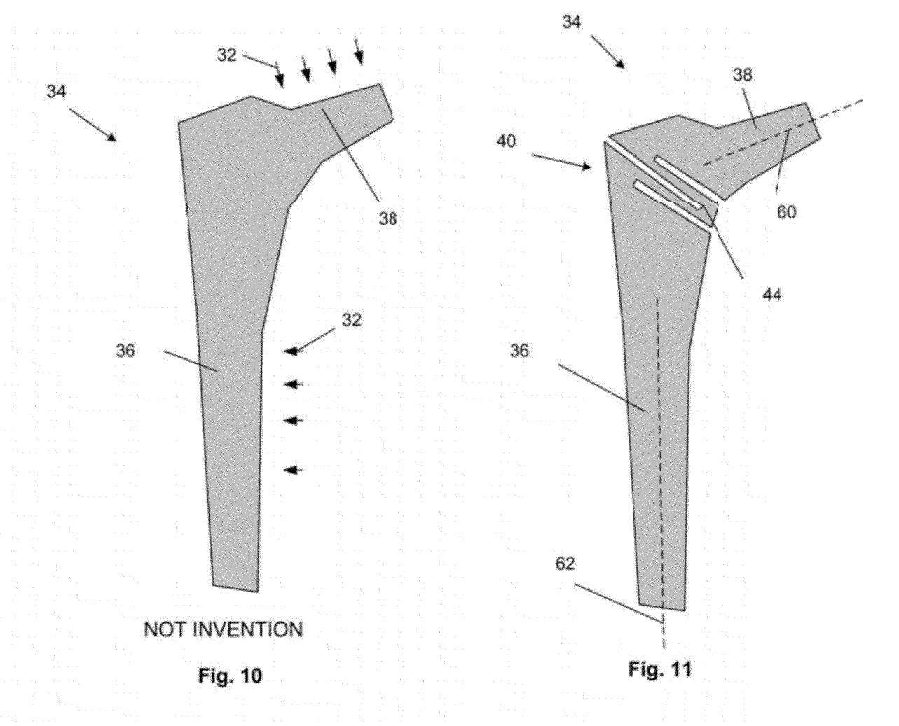

FIG. 9 illustrates that the knee implant femoral component 8 can have an implant medial condyle 54b and an implant lateral condyle 54a. The implant medial condyle 54b and implant lateral condyle 54a can extend from the remainder of the knee implant femoral component 8 (e.g., the component body).

The knee implant femoral component 8 can have an outer layer 56a. The outer layer 56a can be made from a hard metal. The outer layer 56a can be polished or otherwise smoothed. The outer layer 56a can be configured to slide against the meniscus component 12 and/or the tibial component 10.

The outer layer 56a and/or inner shell 56b can be a rigid piece of material.

The knee implant femoral component 8 can have cells 42 and struts 44 that can translate and rotate the outer layer 56a with respect to the inner shell 56b. The cells 42 and struts 44 can extend from the lateral surface of the component to the medial surface

A knee implant femoral component stem 58 can extend perpendicular to the surface and/or in the direction of the concavity of the inner shell 56h. The shell 56b and/or the knee implant femoral component stem 58 can have an ingrowth matrix.

FIG. 11 illustrates that the neck 38 of a hip implant femoral component 34 can have a neck longitudinal axis 60. The femoral stem 36 can have a femoral stem longitudinal axis 62. The femoral stem 36 and/or neck 38 can have one or more ingrowth matrices configured to induce bone growth into the component to anchor the component 34 when implanted at a target site.

The neck 38 can be connected to, attached to, or integral with the femoral stem 36 by an implant spring 40. The implant spring 40 can be configured as any of the springs listed herein. For example, the implant spring 40 can have a cantilevered, U-shaped configuration, similar to the struts 44 shown in FIGS. 8a through 8c. Under load forces 32, the implant spring 40 can resiliently deform the neck 38 with respect to the femoral stem 36. The neck 38 can rotate with respect to the femoral stem 36, resulting in a change in the angle between the neck longitudinal axis 60 and the femoral stem longitudinal axis 62. When the force load 32 is reduced, the angle between the neck longitudinal axis 60 and the femoral stem longitudinal axis 62 can return to the resting angle.

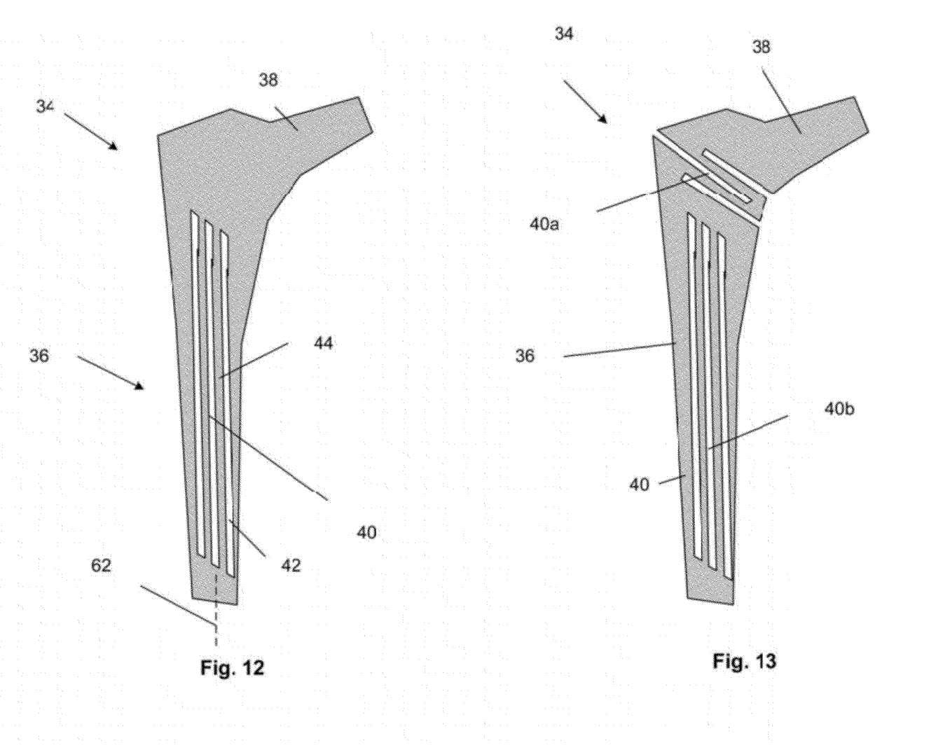

FIG. 12 illustrates that the implant spring 40 can be in the femoral stem. The implant spring 40 can have one or more struts 44 (e.g., formed by cutting slots or cells 42 between the struts) substantially parallel (as shown) and/or perpendicular, and/or at a non-0° and non-90° angle with the femoral stem longitudinal axis 62. The implant spring 40 can minimize stress risers 18 around the femoral stem 36 within the femur 4. Similarly, the implant spring 40 can be in the neck 38.

FIG. 13 illustrates that the implant 2 can have more than one configuration of implant spring 40. For example, the hip implant femoral component 34 can have a first implant spring 40a between the femoral stem 36 and the neck 38 and a second implant spring 40b in the femoral stem 36.

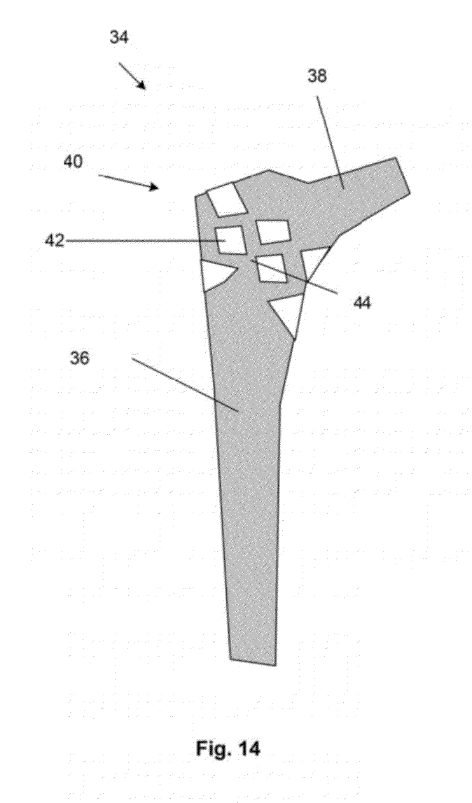

FIG. 14 illustrates that the hip implant femoral component 34 can have an implant spring 40 that can have a number of cells 42 and struts 44. The cells 42 can be polygonal, such as diamond or square shaped, triangular, pentagonal, hexagonal, or combinations thereof. The cells 42 can be rounded, such as circular, oval, or combinations of rounded and polygonal shapes. The neck 38 can resiliently or deformably rotate and/or translate with respect to the femoral stem 36 during use.

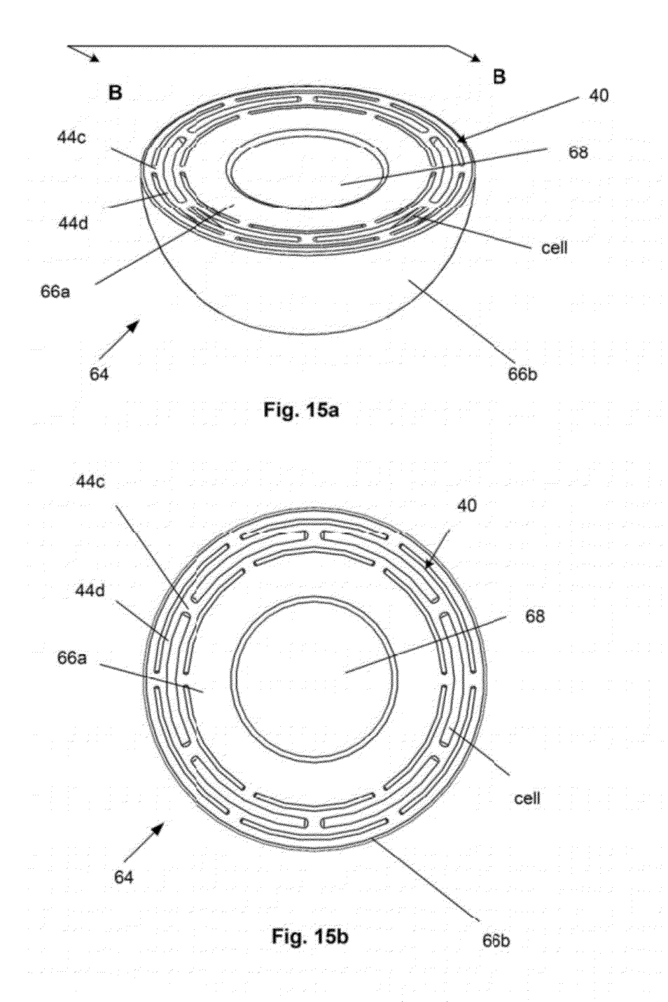

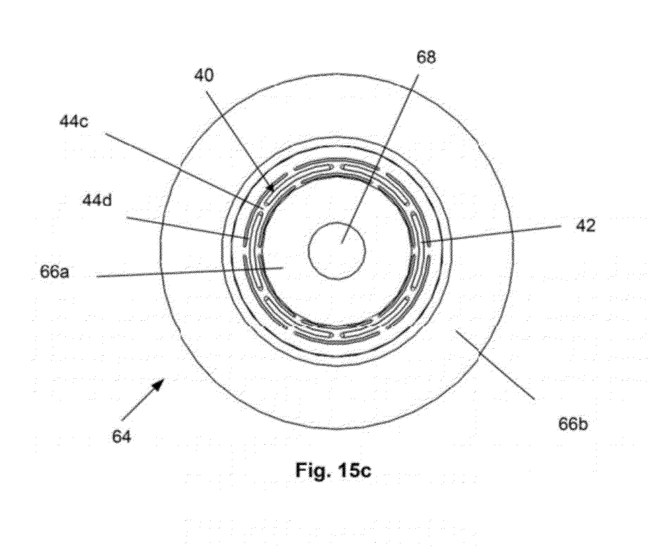

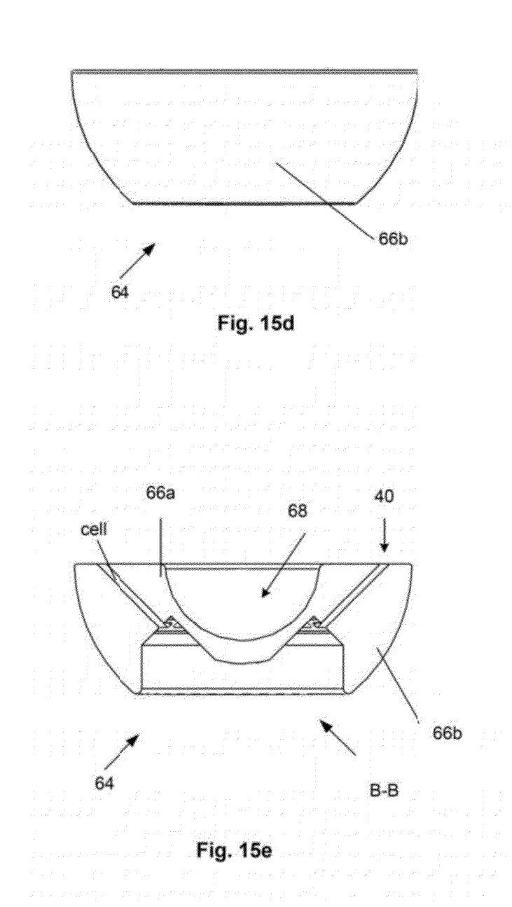

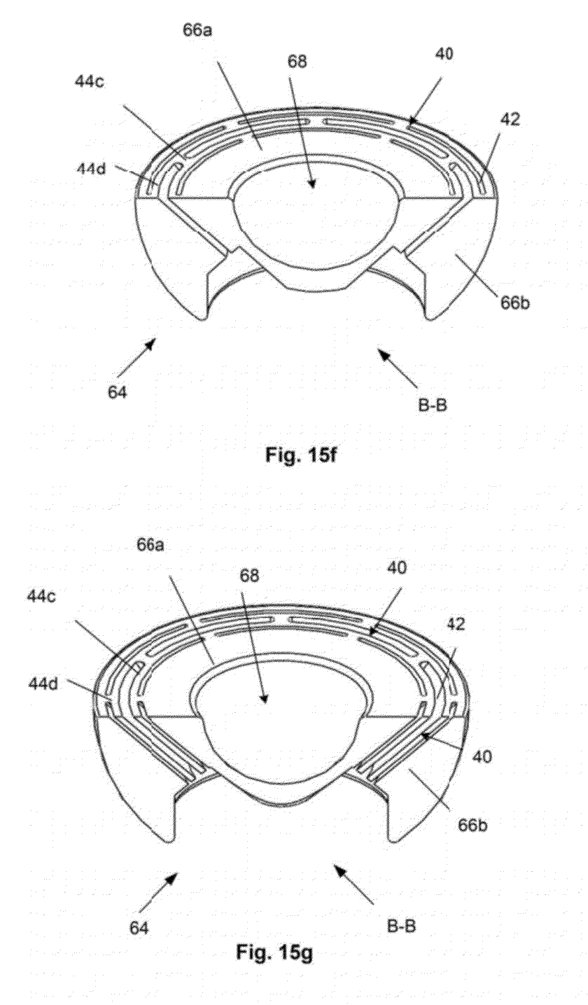

FIGS. 15a through 15g illustrate that acetabulum component 64 can have a seat 68. The seat 68 can be coated with a low-friction material (e.g., PTFE, such as Teflon). The seat 68 can have an artificial cartilage element. The seat 68 can be configured to receive and rotate against the acetabular ball head (not shown) attached to or integral with the hip implant femoral component 34. The acetabulum component 64 can have a substantially hemi-spherical configuration.

The acetabulum component 64 can have an inner layer 66a surrounding the seat 68. The inner layer 66a can be a hard material, such as a metal listed herein, or a soft material, such as an artificial cartilage element, or a hard material lined or coated with a soft material adjacent to the seat 68. The seat 68 can be hemi-spherical. The inner layer 66a can have one or more sub-layers (e.g., a metal radially outer sub-layer and a polymer

The radially outer side of the acetabulum component 64 can have an outer shell 66h. The shell 66b can be a hard material, such as a metal listed herein, and/or have one or more ingrowth matrices. The shell 66b can be connected to, attached to, or integral with (as shown) the inner layer 66a by the implant spring 40.

The implant spring 40 can have radial struts 44c and angular struts 44d that can form cells 42. The cells 42 can be angularly configured between the radial and angular struts 44c and 44d. When force loads 32 are deployed against the inner layer 66a during use, the implant spring 40 can deform. The inner layer 66a can translate and/or rotate with respect to the shell 66b. The translation and/or rotation of the inner layer 66a with respect to the outer shell 66b can minimize stress risers 18 and, for example, reduce damage to the inner layer surface on or adjacent to the seat 68.

FIG. 15g illustrates FIG. 15f at a different cross-section.

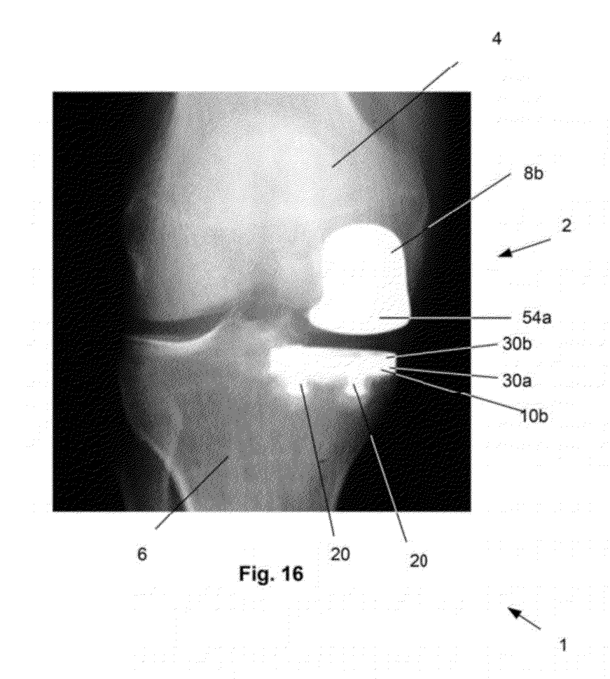

FIG. 16 illustrates an x-ray of the device 2 in use. The material of the medial meniscus component 12 is not visualized in the x-ray, and the spring 40 does not contrast with the surrounding implant material, so the spring 40 is not visibly distinct from the surrounding material.

Any or all elements of the device 2 and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyethylene teraphathalate (PET)/polyester (e.g., DACRON® from E. 1. Du Pont de Nemours and Company, Wilmington, Del.), polypropylene, (PET), polytetrafluoroethylene (PTFE), expanded PTFE (ePTFE), polyether ketone (PFK), polyether ether ketone (PEEK), carbon fiber, PEEK with carbon fiber, poly ether ketone ketone (PEKK) (also poly aryl ether ketone ketone), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), polylactic acid (PLA), polycaprolactone (PCL), polyethyl acrylate (PEA), polydioxanone (PDS), and pseudo-polyamino tyrosine-based acids, extruded collagen, silicone, zinc, echogenic. radioactive, radiopaque materials, a biomaterial (e.g., cadaver tissue, collagen, allograft, autograft, xenograft, bone cement, morselized bone, osteogenic powder, beads of bone) any of the other materials listed herein or combinations thereof. Examples of radiopaque materials are barium sulfate, zinc oxide, titanium, stainless steel, nickel-titanium alloys, tantalum and gold.

Any or all elements of the device 2 and/or other devices or apparatuses described herein, can be, have, and/or be completely or partially coated with agents and/or a matrix a matrix for cell ingrowth or used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, silicone or combinations thereof

The device 2 and/or elements of the device and/or other devices or apparatuses described herein and/or the fabric can be filled, coated, layered and/or otherwise made with and/or from cements, fillers, glues, and/or an agent delivery matrix known to one having ordinary skill in the art and/or a therapeutic and/or diagnostic agent. Any of these cements and/or tillers and/or glues can be osteogenic and osteoinductive growth factors.

Examples of such cements and/or fillers includes bone chips, demineralized bone matrix (DBM), calcium sulfate, coralline hydroxyapatite, biocoral, tricalcium phosphate, calcium phosphate, polymethyl methacrylate (PMMA), biodegradable ceramics, bioactive glasses, hyaluronic acid, lactoferrin, bone morphogenic proteins (BMPs) such as recombinant human bone morphogenetic proteins (rhBMPs), other materials described herein, or combinations thereof.

The agents within these matrices can include any agent disclosed herein or combinations thereof, including radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E, Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

As merely non-limiting examples, the spring 40 in the acetabular cup variation of the device 2 can resist about 2 kN (500 lbs.), and have a resting gap height of from about 0.2 mm (0.009 in.) to about 0.5 mm (0.02 in.). The spring 40 in the tibial component variation of the device 2 can resist about 2 kN (500 lbs.), and have a resting gap height of from about 0.2 mm (0.009 in.) to about 0.5 mm (0.02 in.). The spring 40 in the femoral stem variation of the device 2 can resist about 2 kN (500 lbs.), and have a resting gap height of from about 0.4 mm (0.015 in.) to about 0.8 mm (0.03 in.). The spring 40 in an intervertebral or spinal cage variation of the device 2 can resist about 3.03 kN (681 lbs.), and have a resting gap height of from about 0.5 mm (0.02 in.) to about 1 mm (0.05 in.).

Use of medial and lateral directions herein is exemplary. The directions can be reversed.

It is apparent to one skilled in the art that various changes and modifications can be made to this disclosure, and equivalents employed, without departing from the spirit and scope of the invention. Elements shown with any variation are exemplary for the specific variation and can be used on other variations within this disclosure. Any elements described herein as singular can be pluralized (i.e., anything described as “one” can be more than one). Any species element of a genus element can have the characteristics or elements of any other species element of that genus. The above-described configurations, elements or complete assemblies and methods and their elements for carrying out the invention, and variations of aspects of the invention can be combined and modified with each other in any combination.

We claim:

1. An artificial joint component for use between a first bone and a second bone comprising:
a first element configured to be fixed to the first bone;
a second element; and
a spring integrated with the first plate and the second elements.

2. The component of claim 1, further comprising a stem integral with the first element, wherein the stem is configured to anchor in the first bone.

3. A method of repairing a single axis biological joint having a first bone and a second bone comprising:
replacing the proximal end apart or all of the surface of a first end of a first bone with a first plate resiliently attached to a second plate.

4. The method of claim 4, wherein the single-axis joint comprises a knee joint, and wherein the first bone comprises a tibia and wherein the second hone comprises a femur.

5. The method of claim 4, wherein the single-axis joint comprises an elbow joint.

6. An artificial joint component for use between a first bone and a second bone comprising:
a stem configured to be fixed to the first bone;
a neck; and
a spring.

7. The component of claim 6, wherein the spring is integral with the stem.

8. The component of claim 7, wherein the spring is integral with the neck.

9. The component of claim 6, wherein the spring is integral with the neck.

10. The component of claim 6, wherein the spring is between the spring and the neck.

11. The component of claim 6, wherein the spring is in the stem.

12. A method of repairing a ball-and-socket biological joint having a first bone and a second bone comprising:
inserting a first component into the first bone, wherein the first component comprises a stem, and wherein the stem is inserted into the first bone, and wherein the first component comprises a first spring.

13. The method of claim 12, wherein the first spring is in the stem.

14. The method of claim 12, wherein the first component comprises a neck, and wherein the first spring is between the neck and the stem.

15. The method of claim 14, wherein the first component comprises a second spring, and wherein the second spring is in the stem.

16. The method of claim 4, wherein the ball-and-socket joint comprises a hip, and wherein the first bone comprises a femur.

17. An artificial joint component for use between a first bone and a second bone comprising:
a shell configured to be fixed to the first bone;
an inner layer; and
a spring between the shell and the inner layer.

18. The component of claim 17, wherein the spring comprises a first strut.

19. A method of repairing a ball-and-socket biological joint having a first bone and a second bone comprising:
inserting a first component into the first bone, wherein the first component comprises a shell, and wherein the shell is attached to the first bone, and wherein the first component comprises a first spring.

20. The method of claim 19, wherein the shell has a substantially hemi-spherical shape.

21. The method of claim 19, wherein the ball-and-socket joint comprises a hip, and wherein the first bone comprises a pelvis.

22. The device of claim 1, wherein the first element is a first plate, and wherein the second element is a second plate.
